(19) United States
(12) Patent Application Publication
(10) Pub. No.: US 2003/0108580 A1
(43) Pub. Date: Jun. 12, 2003

(54) Use of granulates based on pyrogenically - produced silicon dioxide in cosmetic compositions

(76) Inventors: Steffen Hasenzahl, Hanau (DE); Riedemann Heike, Mombris (DE); Jurgen Meyer, Stockstadt (DE); Peter Neugebauer, Offenbach (DE)

Correspondence Address: VENABLE, BAETJER, HOWARD AND CIVILETTI, LLP, P.O. BOX 34385, WASHINGTON, DC, 20043-9998, US

(21) Appl. No.: 10/282,124

(22) Filed: Oct. 29, 2002

Related U.S. Application Data
(60) Provisional application No. 60/331,534, filed on Nov. 19, 2001.

(30) Foreign Application Priority Data
Oct. 30, 2001 (DE) 10153077.3

(51) Int. Cl. A61K 7/16; A61K 7/42; A61K 7/04; A61K 7/021; A61K 7/32; A61K 7/06; A61K 7/15; A01N 25/00

(52) U.S. Cl. 424/401; 424/59; 424/63; 424/405; 424/70.1; 424/49; 424/65; 424/73; 424/61

(57) ABSTRACT

The use of granulates based on pyrogenically-produced silicon dioxide in cosmetic compositions, the cosmetic compositions themselves, and an adsorbate of the granulate and at least one other substance, selected from cosmetic active ingredients and auxiliary substances, and the production of such adsorbates, are disclosed.

The present invention relates to the use of granulates of pyrogenic silica in cosmetic compositions. In addition to various other effects, the granulates can perform the function of a carrier for cosmetic active ingredients and/or auxiliary substances.

The use of spherical silicon dioxide particles in cosmetic preparations is known. They are used, for example, in skin care and decorative cosmetics, to produce a smooth and velvety feeling of the skin (“ball bearing effect”); to produce the so-called “soft focus effect”, which gives an even distribution of light falling on the skin and thus reduces the appearance of wrinkles and flaws in the skin; to improve the resistance of make up, by adsorbing the tallow produced by the skin or as carriers for cosmetic active ingredients, cosmetic oils and colourants.

U.S. Pat. No. 4,837,011 discloses the use of spherical silicon dioxide particles with an average particle size of 6 to 20 μm in a cosmetic powder. The silicon dioxide particles used are obtained according to JP OS 61174103 by spraying a dispersion of silica sol and silica gel in a gas stream followed by drying. The silicon dioxide-containing powder is characterised by excellent adhesion to the skin and can be applied easily and smoothly.

EP OS 0 679 382 discloses spherical silicon dioxide particles with a preferred average particle size of 1 to 50 μm, which contain a UV-reflecting metal oxide and are coated with N-lauryl-L-lysine, and also cosmetic compositions containing these silicon dioxide particles. The cosmetics are easy to apply, make the skin feel soft and have good skin adhesion and water repellence. Both porous and non-porous silicon dioxide particles can be used, various methods being disclosed for the production of the porous particles. For example, fine liquid droplets of a silicate solution can be gelled with carbon dioxide in a non-polar organic solvent in the presence of a surfactant, washed and dried; fine droplets of a silicic acid ester can also be formed in a solvent, which are then gelled with ammonia or similar; or spherical silicon dioxide particles are produced by spray-drying silica sols. Non-porous spherical silicon dioxide particles can be obtained e.g. by tempering spherical or irregularly-shaped silicon dioxide particles in a gas stream at raised temperature.

WO OS 96/17583 relates to a cosmetic preparation, which contains spherical silicon dioxide particles with an average particle size of 3 to 16 μm. The silicon dioxide particles here serve on the one hand as carriers for moisture-containing agents, on the other to absorb excess tallow from the skin. No process for the production of silicon dioxide particles is disclosed.

DE OS 199 29 109 provides spherical silicon dioxide particles with a diameter of 50 nm to 50 μm and a coating of a metal oxide or a metal oxide and a colouring material. In addition to the use of these coated silicon dioxide particles as colour pigments in paints, printing inks and plastics, their use in cosmetic formulations is also disclosed. The coated silicon dioxide particles have excellent colouring power, good reproducibility of colouring and, in cosmetic applications, give the skin a soft and velvety feel.

DE OS 198 42 134 relates to similar spherical silicon dioxide particles with a diameter of less than 50 μm, which are coated either with titanium dioxide and silicon dioxide or with titanium dioxide and Iron (III)-oxide. These silicon dioxide particles are used in cosmetic formulations on the one hand to give the skin a natural appearance, but on the other to make wrinkles largely invisible. This is achieved by diffusing the reflected and transmitted light.

The spherical silicon dioxide particles from DE OS 199 29 109 and DE OS 198 42 134 are produced by hydrolysis of organic or inorganic silicon compounds in an emulsion process, as disclosed for example in DE OS 21 55 281, DE OS 26 10 852, GB OS 1 141 929 and EP OS 0 162 716.

Cosmetic exfoliating products to remove skin impurities and dead skin cells, which contain a particle-shaped material that can be rubbed away, with a particle size in the range 0.03 to 3 mm, are disclosed in WO OS 94/12151. The material that can be rubbed away may be for example agglomerates of precipitated silica with a primary particle size of 0.001 to 0.2 μm.

The disadvantage of all the silicon dioxide particles mentioned above, which are used in cosmetic peperations, is that they are laborious and costly to produce. Thus either expensive starting compounds such as silicic acid esters or silica sols are used, or silicates, which then often produce products of insufficient purity for the desired application—they often still contain considerable quantities of salts—so that laborious washing is required.

The object of the present invention is therefore to provide spherical silicon dioxide particles for use in cosmetic compositions, which do not have the stated disadvantages and also fulfil the exacting requirements of the cosmetics industry for purity and product safety.

The object is achieved by the use of a granulate based on pyrogenically-produced silicon dioxide in a cosmetic composition. The present invention also provides a cosmetic composition that contains a granulate based on pyrogenically-produced silicon dioxide and conventional cosmetic constituents. The present invention further relates to an adsorbate consisting of a granulate based on pyrogenically-produced silicon dioxide and at least one other substance, selected from cosmetic active ingredients and auxiliary substances, and the production of such adsorbates.

The granulate based on pyrogenically-produced silicon dioxide preferably has an average grain size of 10 to 120 μm and a BET-surface of 40 to 400 m²/g (determined to DIN 66 131 with nitrogen).

More preferably, the silicon dioxide granulate also has the following physical-chemical reference data, determined as disclosed in EP PS 0 725 037:

| | |
|---|---|
| Pore volume: | 0.5 to 2.5 ml/g |
| Pore size distribution: | less than 5% of the total pore |
| | volume has a pore diameter of less |
| | than 5 nm, the rest meso- and macro |
| | pores |
| pH-value: | 3.6 to 8.5 |
| Tamped density: | 220 to 700 g/l. |

A granuate suitable for the use according to the invention and the production thereof is disclosed for example in EP OS 0 727 037.

The granulate preferably contains meso- and macro pores, the volume of the meso pores constituting 10 to 80% of the total volume. The particle size distribution of the granulate is preferably 80 vol. % larger than 8 μm and 80 vol. % smaller than 96 μm. In a preferred embodiment of the invention, the proportion of pores smaller than 5 μm can be no more than 5 % in relation to the total pore volume.

The granulate used according to the invention can be produced, for example, by dispersing in water pyrogenically-produced silicon dioxide, preferably silicon dioxide produced from silicon tetrachloride by flame hydrolysis, spray drying it and then optionally tempering the granulate obtained at a temperature of 150 to 1,100° C. for a period of 1 to 8 h.

The dispersion in water preferably has a concentration of silicon dioxide of 5 to 25 wt. %, more preferably 5 to approx. 19.9 wt. %. Spray drying can be carried out at a temperature of 200 to 600° C., using disk or nozzle atomisers. The granulate can be tempered both in a fixed bed, such as for example in chamber ovens, or in a fluid bed such as for example a rotary kiln.

The pyrogenic silicon dioxide used as a starting compound is produced by jetting a volatile silicon compound into an oxyhydrogen flame of hydrogen and air. In most cases, silicon tetrachloride is used. This substance hydrolyses under the influence of the water formed in the oxyhydrogen reaction to form silicon dioxide and hydrochloric acid.

After leaving the flame, the silicon dioxide enters a so-called coagulation zone, in which the silicon dioxide primary particles and primary aggregates agglomerate. The product present at this stage as a kind of aerosol, is separated in cyclones from the gaseous companion substances and then post-treated with moist hot air. This process reduces the residual hydrochloric acid content to less than 0.025%.

The granulates based on pyrogenically-produced silicon dioxide can also be silanised. The carbon content of the granulate is then preferably 0.3 to 15.0 wt. %. Halogen silanes, alkoxysilanes, silazanes and/or siloxanes can be used for silanation.

The following substances in particular can be used as halogen silanes:

Halogen organosilanes of the type X₃Si (CₙH₂ₙ₊₁)

X=Cl, Br

n=1-20

Halogen organosilanes of the type X₂(R′)Si(CₙH₂ₙ₊₁)

X=Cl, Br

R′=alkyl

n=1-20

Halogen organosilanes of the type X(R′)₂Si(CₙH₂ₙ₊₁)

X=Cl, Br

R′=alkyl

n=1-20

Halogen organosilanes of the type X₃Si(CH₂)ₘ—R′

X=Cl, Br

m=0.1-20

R′=alkyl, aryl (e.g. —C₆H₅) —C₄F₉, —OCF₂—CHF—CF₃, —C₆F₁₃, —O—CF₂—CHF₂ —NH₂, —N₃, —SCN, —CH═CH₂, —OOC(CH₃)C═CH₂ —OCH₂—CH(O)CH₂

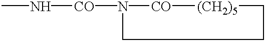

—NH—COO—CH₃, —NH—COO—CH₂—CH₃, —NH—(CH₂)₃Si(OR)₃ —Sₓ—(CH₂)₃Si(OR)₃

Halogen organosilanes of the type (R)X₂Si(CH₂)ₘ—R′

X=Cl, Br

R=alkyl

m=0.1-20

R′=alkyl, aryl (e.g. —C₆H₅) —C₄F₉, —OCF₂—CHF—CF₃, —C₆F₁₃, —O—CF₂—CHF₂ —NH₂, —N₃, —SCN, —CH═CH₂, —OOC(CH₃)C═CH₂ —OCH₂—CH(O)CH₂

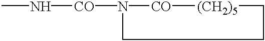

—NH—COO—CH₃, —NH—COO—CH₂—CH₃, —NH—(CH₂)₃Si(OR)₃ —Sₓ—(CH₂)₃Si(OR)₃

Halogen organosilanes of the type (R)₂X Si(CH₂)ₘ—R′

X=Cl, Br

R=alkyl

m=0.1-20

R′=alkyl, aryl (e.g. —C₆H₅) —C₄F₉, —OCF₂—CHF—CF₃, —C₆F₁₃, —O—CF₂—CHF₂ —NH₂, —N₃, —SCN, —CH═CH₂, —OOC(CH₃)C═CH₂ —OCH₂—CH(O)CH₂

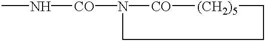

—NH—COO—CH₃, —NH—COO—CH₂—CH₃, —NH—(CH₂)₃Si(OR)₃ —Sₓ—(CH₂)₃Si(OR)₃

The following substances can be uses in particular as alkoxysilanes:

Organosilanes of the type (RO)₃Si(CₙH₂ₙ₊₁)

R=alkyl

n=1-20

Organosilanes of the type R′ₓ(RO)_{y}Si(CₙH₂ₙ₊₁)

R=alkyl

R′=alkyl

n=1-20

x+y=3

x=1.2

y=1.2

Organosilanes of the type (RO)₃Si(CH₂)ₘ—R′

R=alkyl

m=0.1-20

R′=alkyl, aryl (e.g. —C₆H₅) —C₄F₉, OCF₂—CHF—CF₃, —C₆F₁₃, —O—CF₂—CHF₂ —NH₂, —N₃, —SCN, —CH═CH₂, —OOC(CH₃)C═CH₂ —OCH₂—CH(O)CH₂

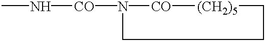

—NH—COO—CH₃, —NH—COO—CH₂—CH₃, —NH—(CH₂)₃Si(OR)₃ —Sₓ—(CH₂)₃Si(OR)₃

Organosilanes of the type (R″)ₓ(RO)_{y}Si(CH₂)ₘ—R′

R″=alkyl

x+y=2

x=1.2

y=1.2

R′=alkyl, aryl (e.g. —C₆H₅) —C₄F₉, —OCF₂—CHF—CF₃, —C₆F₁₃, —O—CF₂—CHF₂ —NH₂, —N₃, —SCN, —CH═CH₂, —OOC(CH₃)C═CH₂ —OCH₂—CH(O)CH₂

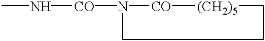

—NH—COO—CH₃, —NH—COO—CH₂—CH₃, —NH—(CH₂)₃Si(OR)₃ —Sₓ—(CH₂)₃Si(OR)₃

Silane Si 108 [(CH₃O)₃—Si—C₈H₁₇] trimethoxyoctylsilane is preferably used as a silanising agent.

The following substances in particular can be used as silazanes:

Silazanes of the type:

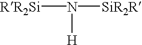

R=alkyl

R′=alkyl, vinyl

and also Hexamethyldisilazane, for example.

The following substances in particular can be used as siloxanes:

Cyclic polysiloxanes of the type D3, D4, D5, e.g. octamethylcyclotetrasiloxane=D4

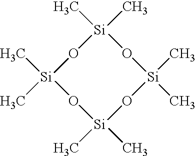

polysiloxanes e.g. silicon oils of the type:

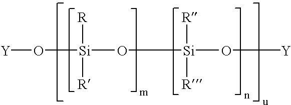

R=alkyl, aryl, (CH₂)ₙ—NH₂, H

R′=alkyl, aryl, (CH₂)ₙ—NH₂, H

R″=alkyl, aryl, (CH₂)ₙ—NH₂, H

R′″=alkyl, aryl, (CH₂)ₙ—NH₂, H

Y=CH₃, H, CₙH₂ₙ₊₁ with n=1-20

Y=Si(CH₃)₃, Si(CH₃)₂H Si(CH₃)₂OH, Si(CH₃)₂(OCH₃) Si(CH₃)₂(CₙH₂ₙ₊₁) with n=1-20

m=0, 1, 2, 3, . . . ∞

n=0, 1, 2, 3, . . . ∞

u=0, 1, 2, 3, . . . ∞

Silanation can be carried out by spraying the granulate with a silanising agent, which can optionally be dissolved in an organic solvent, such as for example ethanol, and then thermally treating the mixture at a temperature of 105 to 400° C. for a period of 1 to 6 h.

An alternative method of silanising granulates can be used, in which the granulate is treated with the silanising agent in vapour form and then the mixture is thermally treated at a temperature of 200 to 800° C. for a period of 0.5 to 6 h. The thermal treatment can take place in a protective gas, such as for example nitrogen.

Silanation can be carried out in heatable mixers and driers with spray devices, continuously or in batches. Suitable

By varying the substances used, the spraying, tempering and silanation conditions, the physical-chemical parameters of the granulates, such as specific surface, the particle size distribution, the pore volume, the tamped density and the silanol group concentration, pore distribution and pH value can be changed within the given limits.

The granulates of pyrogenic silicon dioxide can be used according to the invention in cosmetic compositions of any consistency, e.g. in powders, liquids, foams, sprays, gels, creams, salves, pastes, sticks or tablets. Accordingly, the cosmetic compositions may be single- or multi-phase systems such as for example emulsions, suspensions or aerosols.

The cosmetic composition may be, for example, a soap; a synthetic “soapless” soap; a liquid washing or shower preparation; a bath additive; a make-up remover; an exfoliating preparation; a skin cream; a skin lotion; a face mask; a footcare product; a sun protection product; a skin tanning product; a de-pigmenting product; an insect repellent; a wet-shave product, such as a stick, cream, gel or foam; a pre-shave product; an after-shave care product; a depilatory product; a toothpaste; a hair shampoo; a hair care product, such as a hair mask, a rinse or a conditioner; a permanent wave product; a smoothing product, a hair styling product, such as a setting lotion, a hair spray, a hair lacquer, a hair gel or a hair wax; a hair colourant, such as a bleaching product, a hair colouring product, a tint or a colour fixer; a deodorant or an anti-perspirant, such as a stick, roll-on, lotion, powder or spray; a face make-up, such as a tinted day cream, a cream-to-powder foundation, a face powder, a cream foundation or a blusher; an eye make up, such as an eyeshadow, a mascara, a kohl pencil, an eyeliner or an eyebrow pencil; a lip care product; a decorative lip care product, such as a lipstick, a lip gloss or a lipliner pencil; a nail care product, such as a nail polish, a nail polish remover, a cuticle remover, a nail hardener or a nail care cream.

The present invention also provides a cosmetic composition, containing the previously-defined silicon dioxide granulates and at least one constituent selected from absorbents, astringents, antimicrobial substances, antioxidants, anti-perspirants, anti-foam agents, anti-dandruff active ingredients, antistatic agents, binders, biological additives, bleaching agents, chelating agents, deodorants, emollients, emulsifiers, emulsion stabilisers, depilatory agents, colours, moisture-containing agents, film formers, perfumes, flavourings, hair colourants, preservatives, anti-corrosion agents, cosmetic oils, solvents, mouth care substances, oxidation agents, vegetable constituents, buffering agents, reducing agents, abrasives, detergents, propellents, opacity agents, UV filters and absorbers, denaturing agents, viscosity regulators and vitamins.

Depending on the cosmetic composition, in which the silicon dioxide granulates are used, they may have various functions. They serve for example, to improve the feel of the skin (ball bearing effect), adhesion to the skin and ease of application. Furthermore, the long-term stability of decorative cosmetics such as make-up, is improved by the adsorption of skin tallow and oil. Also in decorative cosmetics, they improve the appearance of wrinkles by means of optimum, even distribution of light. In skin and hair cleansing products, the silicon dioxide granulates can act as abrasives. They are also suitable for concealing or absorbing characteristic or even unpleasant odours of cosmetic consituents, which could otherwise not be used. A further function is the fixing, or slow and controlled release, of highly volatile substances, e.g. essential oils, aromas and perfumes. In many cosmetic compositions they also act as fillers. Hydrophobic silicon dioxide granulates i.e. silanised granulates, are particularly suitable for the production of waterproof cosmetics.

However, the silicon dioxide granulates used according to the invention preferably act as carriers for cosmetic active ingredients and/or auxiliary substances. The present invention thus also relates to an adsorbate of the silicon dioxide granulate mentioned previously and at least one of these substances.

The expression “adsorbate”, as used here, encompasses not only the adsorption of a substance on the surface of the silicon dioxide, but also in the pores, and the “insertion” into the voids between the grains. “Adsorbate” can also mean that silicon dioxide granulate or fragments thereof, coats solid particles or liquid droplets of the substance. In the latter case, the attractive forces between the particles or droplets are reduced and, for example, the flow behaviour is improved or droplets are prevented from flowing together.

Examples of cosmetic active ingredients and auxiliary substances are the constituents of a cosmetic composition mentioned previously.

A cosmetic active ingredient according to this invention is deemed, as defined by Umbach (1995), to mean a substance in cosmetic preparations, which, under application conditions, has a physical, physical/chemical, chemical, biochemical and/or subject-related action, influencing inter alia the physiology and/or function of the skin or mucous membrane and their appendages, as well as the teeth, but excluding any significant effect on the organism. Examples of cosmetic active ingredients that can be adsorbed onto the silicon dioxide granules are vitamins; moisture-containing agents such as polyalcohols, ceramides and compounds similar to ceramides; physical and chemical UV filters and astringents.

Of the cosmetic auxiliary substances, cosmetic oils, perfumes, flavourings or colours are preferably adsorbed onto the silicon dioxide granulate. The perfumes and flavourings may be either of natural, i.e. vegetable or animal, or synthetic, i.e. fully- and semi-synthetic, origin.

Examples of vegetable perfumes are essential oils and resinoids. Animal perfumes include e.g. musk, civet, castoreum and ambergris. The fully-synthetic perfumes include both those having a natural equivalent and purely invented compositions. Semi-synthetic perfumes are understood to be those isolated from natural perfumes and then chemically altered.

The colourants can also be natural or synthetic; they may be organic or inorganic compounds.

The quantity ratio of substance to silicon dioxide granulate in the adsorbate may be selected at will, depending on the properties of the substance and the requirements of the end product. However, 0.001 to 200 g substance per 100 g silicon dioxide granulate is preferably used, and in particular 10 to 150 g.

An example of a process for the production of the adsorbate according to the invention comprises:

(a) melting of the substance(es) to be adsorbed, selected from cosmetic active ingredients and auxiliary substances, or distribution, i.e. dissolution, suspension or emulsification of these substances in a solvent;

(b) mixing of the granulate based on pyrogenically-produced silicon dioxide with the mixture from step (a); and

(c) optionally, removal of the solvent.

“Solvents” also include mixtures of several different solvents. It is also understood that substances that are liquid at room temperature can be subjected to the mixing in step (b) without any prior processing, as in this case the “melting process” has already taken place. The mixing step (b) can take place either by adding the mixture from step (a) to the silicon dioxide granulate, e.g. by spraying, or vice-versa. In both cases, addition can take place in one or several portions. The mixing time in step (b) depends primarily on the adsorption behaviour of the substance to be adsorbed on the silica surface. If a solvent is present, step (a) and step (b) are carried out at a temperature between the freezing and boiling point of the solvent. The optional excess solvent is removed in step (c), preferably at increased temperature and/or reduced pressure.

The removal of the solvent in step (c) can also be carried out either by spray- or fluid-bed drying, in which case the moulding process takes place simultaneously. Accordingly, the moulding process for a granulate-containing melt may be extrusion.

The advantages of the granulates of pyrogenic silicon dioxide used according to the invention are firstly that they are simple and inexpensive to produce and secondly that they are extremely pure.

The invention will now be explained in more detail with the aid of examples.

Reference Example: Production of a granulate based on pyrogenically-produced silicon dioxide.

The pyrogenically-produced silicon dioxide AEROSIL 90, commercially available from Degussa AG, is used as a starting compound.

The pyrogenically-produced silicon dioxide is dispersed in fully-desalinated water. A dispersing unit is used for this, which operates by the rotor/stator principle. The suspension formed is spray-dried. The finished product is separated by filter or cyclone. The spray granulate is tempered in a muffle kiln.

The production parameters are given in Table 1.

TABLE 1

| Starting-SiO₂ | | AEROSIL 90 |
|---|---|---|
| Spray drying data | | |
| Quantity H₂O | (kg) | 100 |
| Quantity SiO₂ | (kg) | 1.5 |
| Atomisation with | | single-fluid |
| | | nozzle |
| Operating temperature | (° C.) | 358 |
| Outlet air temperature | (° C.) | 105 |
| Separation | | filter |
| Physical/Chemical Data | | |
| BET-Surface | (m²/g) | 87 |
| Grain size d⁵^{⁰} | (μm) | 25 |
| Tamped volume | (g/l) | 258 |
| pH-value | | 4.7 |
| Carbon content % | | — |

### EXAMPLE 1

Tinted Day Cream With AEROSIL 90 Granulate

A tinted day cream was produced according to the recipe in Table 2 and the method that follows, using 10 wt. % of the granulate produced in the reference example from AEROSIL 90.

TABLE 2

| | Constituent | INCI-Name | Wt. % | 200 g |
|---|---|---|---|---|
| A | AEROSIL 90 | Silica | 10.00 | 20.00 |
| | granulate | | | |
| | Veegum HV | Magnesium Aluminium | 1.00 | 2.00 |
| | (Vanderbilt) | Silicate | | |
| | Euxyl K 400 | Methyldibromo | 0.10 | 0.20 |
| | (Schülke + Mayer) | Glutaronitrile, | | |
| | | Phenoxyethanol | | |
| | Sorbitol F liquid | Sorbitol | 3.00 | 6.00 |
| | 1.02993 (Caelo) | | | |
| | Water | Aqua (Water) | 48.44 | 96.88 |
| B | Methyl-4-hydroxy- | Methylparaben | 0.18 | 0.36 |
| | benzoate 1.30174 | | | |
| | (Merck) | | | |
| | Propyl-4-hydroxy- | Propylparaben | 0.08 | 0.16 |
| | benzoate 1.30173 | | | |
| | (Merck) | | | |
| | 1,2-Propanediol | Propylene Glycol | 3.00 | 6.00 |
| | 1.30140 (Merck) | | | |
| C | Arlacel 165 (Uniqema) | Glyceryl Stearate, | 5.00 | 10.00 |
| | | PEG-100 Stearate | | |
| | Lanette O (Caelo / | Cetearyl Alcohol | 1.50 | 3.00 |
| | Henkel) | | | |
| | Tegosoft CT | Caprylic Capric | 7.00 | 14.00 |
| | (Goldschmidt) | Triglyceride | | |
| | Shea Butter | Butyrospermum | 2.00 | 4.00 |
| | (Karlshamns) | Parkii (Shea | | |
| | | Butter) | | |
| | Tegosoft CI | Cetearyl | 7.00 | 14.00 |
| | (Cetiol SN) | Isononanoate | | |
| | (Goldschmidt) | | | |
| | Eutanol G (Caelo / | Octyldodecanol | 7.50 | 15.00 |
| | Henkel) | | | |
| | Emulgade PL 68/50 | Cetearyl Glucoside, | 2.00 | 4.00 |
| | (Henkel) | Cetearyl Alcohol | | |
| D | Perfume | Parfum | 0.20 | 0.40 |
| | Dow Corning 345 | Cyclomethicone | 2.00 | 4.00 |
| | (Dow Corning) | | | |
| | Citric acid (Merck) | Citric Acid | +UZ,24/32 | |
| | | | 100.00 | 200.00 |

Method:

The AEROSIL 90 granulate and the other components of phase A were added to the aqueous dispersion of Veegum in water whilst stirring. The resulting mixture was then heated to 80° C. The components of phase B were also mixed and heated until a clear mixture formed. Phase B was then added to phase A whilst stirring. The components of phase C were also mixed, heated to 80° C. and quickly added to the mixed phases A and B. The resulting mixture was homogenised for one minute. It was allowed to cool to 40° C. whilst stirring (blade mixer 500 min⁻¹) and phase D was then added.

The cream thus produced gave a pleasent feeling to the skin and could be applied to the skin easily. Viscosity (Brookfield, Speed 01, T-E Spindle, 26° C.): 1545 mpa.s

### EXAMPLE 2

Day Cream Containing Vitamin E

50.0 g of the granulate produced in the reference example from AEROSIL 90 were placed into a tall 600 ml beaker and 50.0 g vitamin E acetate (BASF) were added in portions with a spatula. The granulate quickly absorbed the oily liquid, did not produce dust and did not become electrostatically charged. The entire quantity of vitamin E acetate was processed within ten minutes. The dry mixture was then sifted through a screen with a mesh width of 0.75 mm and allowed to stand overnight.

The flow mark and discharge cone height of the powdery adsorbate were then determined as described in the pigments literature series, No. 31 “AEROSIL for the improvement of the flow behaviour of powdery substances” from Degussa AG, bulk and tamped density were determined according to DIN standard 66131.

Table 3: Characterisation of the Vitamin E Acetate Adsorbate

| | |
|---|---|
| Flow mark | 1 |
| Discharge cone height (cm) | 1.35 |
| Bulk density (g/l) | 431 |
| Tamped density (g/l) | 500 |

A cream analogous to example 1 was produced with the powdery adsorbate thus produced, using 10 wt. % of the vitamin E acetate adsorbate instead of 10 wt. % of the pure AEROSIL 90 granulate.

The cream thus produced gave a pleasant feeling to the skin and was easy to apply to the skin. Viscosity (Brookfield, Speed 01, T-E Spindle, 26° C.): 13600 mpa.s.

### EXAMPLE 3

Cream-To-Powder Foundation

A cream-to-powder foundation was produced according to the recipe from Table 4 and the instructions that follow, using 10 wt. % of the granulate produced in the reference example from AEROSIL 90.

TABLE 4

| | Constituents | INCI-Name | wt. % |
|---|---|---|---|
| A | Tegosoft OP | Octyl Palmitate | 29 |
| | (Goldschmidt) | | |
| | Amerchol L-101 (Amerchol | Mineral Oil, Lanolin | 2.5 |
| | Corp.) | Alcohol | |
| | Carnauba wax (Caelo) | Carnauba | 1.3 |
| | Paracera W80 (Paramelt) | Ceresin | 2.0 |
| | Tegosoft CH | Hydrogenated Castor Oil | 3.5 |
| | (Goldschmidt) | | |
| | Dow Corning 556 (Dow | Phenyl Trimethicone | 9.5 |
| | Corning) | | |
| | Arlacel 83 (Uniqema) | Sorbitan Sesquioleate | 0.5 |
| | DL-alpha- | Tocopheryl acetate | 0.5 |
| | Tocopherolacetate | | |
| | 5.00952 (Merck) | | |
| | Propyl-4-hydroxybenzoate | Propylparaben | 0.2 |
| | 1.30173 (Merck) | | |
| | Oxynex K liquid 1.08324 | PEG-8, Tocopherol, Ascorbyl | 0.1 |
| | (Merck) | Palmitat, Ascorbic Acid; | |
| | | Citric Acid | |
| B | AEROSIL 90 granulate | Silica | 10.0 |
| | Amihope LL | Lauroyl Lysine | 1.5 |
| | (Lehmann + Voss/ | | |
| | Ajinomoto) | | |
| | Dry Flow PC (National | Aluminium Starch, | 14.4 |
| | Starch + Chemical) | Octenylsuccinate | |
| | Extender W 1.17311 (Fa. | Mica, CI 77891 (Titanium | 12.0 |
| | Merck) | Dioxide) | |
| | Microna Matte Yellow | Mica, CI 77492 (Iron | 4.8 |
| | 1.17436 (Merck) | Oxides) | |
| | Microna Matte Black | Mica, CI 77499 (Iron | 0.6 |
| | 1.17437 (Merck) | Dioxide) | |
| | Microna Matte Red | Mica, CI 77491 (Iron | 0.8 |
| | 1.17435 (Merck) | Oxide) | |
| | Microna Matte Orange | Mica, CI 77491 (Iron | 0.4 |
| | 1.17449 (Merck) | Oxide) | |
| | Talk 1.08070 (Merck) | Talc | 6.4 |
| | | | 100 |

Method:

The components of Phase A were mixed with each other and heated to 85° C. whilst stirring producing a clear, liquid phase. The components of Phase B were also mixed and added to the molten Phase A whilst stirring. The liquid mixture was then poured into a mould at 80° C. and left there to cool.

After cooling to room temperature, a solid, soft product was obtained that can be applied to the skin easily with a sponge or brush. Resistance and adhesion to the skin were very good.

1. Use of a granulate based on pyrogenically-produced silicon dioxide in a cosmetic composition.

2. Use according to claim 1, characterised in that the granulate has an average grain diameter of 10 to 120 μm and a BET-surface of 40 to 400 m²/g (determined to DIN 66 131 with nitrogen).

3. Use according to claim 1 or 2, characterised in that the cosmetic composition is in the form of a powder, liquid, foam, spray, gel, cream, salve, paste, stick or tablet.

4. Use according to one of claims 1 to 3, characterised in that the cosmetic composition is a soap, synthetic “soapless” soap, liquid washing or shower preparation, bath additive, make-up remover, exfoliating product, skin cream, skin lotion, face mask, footcare product, sun protection cream, skin tanning product, de-pigmenting product, insect repellent, wet-shave product, pre-shave product, after-shave care product, depilatory product, toothpaste, hair shampoo, hair care product, permanent wave product, smoothing product; hair styling product, hair colouring product, deodorant, anti-perspirant, face make-up, eye make-up, lip care product, decorative lip care product or nail care product.

5. Use according to claim 4, characterised in that the wet-shave product is a stick, cream, gel, or foam; the hair care product is a hair mask, rinse or conditioner; the hair styling product is a setting lotion, hairspray, hair lacquer, hair gel or hair wax; the hair colouring product is a bleaching agent, a hair colourant, a tint or a colour fixing agent; the deodorant or anti-perspirant is a stick, roll-on, lotion, powder or spray; the face make-up is a tinted day cream, a cream-to-powder foundation, a face powder, a cream foundation or a blusher; the eye make-up is an eyeshadow, mascara, kohl pencil, eyeliner or eyebrow pencil; the decorative lip care product is a lipstick, lipgloss or lipliner pencil and the nail care product is a nail polish, nail polish remover, cuticle remover, nail hardener or a nail care cream.

6. Use according to one of claims 1 to 5, characterised in that the granulate acts as a carrier for cosmetic active ingredients and/or auxiliary substances.

7. Cosmetic composition containing a granulate based on pyrogenically-produced silicon dioxide and at least one constituent selected from:
Absorbents, astringents, antimicrobial substances, antioxidants, anti-perspirants, anti-foam agents, anti-dandruff active ingredients, antistatic agents, binders, biological additives, bleaching agents, chelating agents, deodorants, emolients, emulsifiers, emulsion stabilisers, depilatory agents, colourants, moisture-containing agents, film-formers, perfumes, flavourings, hair colourants, preservatives, anti-corrosion agents, cosmetic oils, solvents, mouth care products, oxidation agents, vegetable constituents, buffering substances, reducing agents, abrasives, detergents, propellants, opacity agents, UV-filters and absorbers, denaturing agents, viscosity regulators and vitamins.

8. Cosmetic composition according to claim 7, characterised in that the granulate has an average grain diameter of 10 to 120 μm and a BET-surface of 40 to 400 m²/g (determined to DIN 66 131 with nitrogen).

9. Adsorbate from a granulate based on pyrogenically-produced silicon dioxide and at least one other substance, selected from cosmetic active ingredients and auxiliary agents.

10. Adsorbate according to claim 9, characterised in that the granulate has an average grain size of 10 to 120 μm and a BET-surface of 40 to 400 m²/g (determined to DIN 66 131 with nitrogen).

11. Adsorbate according to claim 9 or 10, characterised in that the cosmetic active ingredients and auxiliary substances are selected from the substances listed in claim 7.

12. Process for the production of an adsorbate according to one of claims 9 to 11 comprising:
(a) melting of the substance(s) to be adsorbed, selected from cosmetic active ingredients and auxiliaries, or distribution of these in a solvent;
(b) mixing of the granulate based on pyrogenically-produced silicon dioxide with the mixture from step (a); and
(c) optionally, removal of the solvent.
